(19) United States
(12) Patent Application Publication
(10) Pub. No.: US 2025/0327798 A1
(43) Pub. Date: Oct. 23, 2025

(54) NOVEL DIAGNOSTIC METHOD FOR DETECTION OF MYASTHENIA GRAVIS

(71) Applicants: Richard Fahlman, Edmonton (CA); Ramanaguru Piragasam, Edmonton (CA); Faraz Hussain, Edmonton (CA); Zaeem Siddiqi, Edmonton (CA)

(72) Inventors: Richard Fahlman, Edmonton (CA); Ramanaguru Piragasam, Edmonton (CA); Faraz Hussain, Edmonton (CA); Zaeem Siddiqi, Edmonton (CA)

(21) Appl. No.: 18/857,429

(22) PCT Filed: Apr. 19, 2023
(86) PCT No.: PCT/CA2023/050526

§ 371 Date: Oct. 16, 2024

Related U.S. Application Data
(60) Provisional application No. 63/332,494, filed on Apr. 19, 2022.

(30) Foreign Application Priority Data
Apr. 19, 2023 (WO) PCT/CA2023/050526

(51) Int. Cl. G01N 33/564; A61K 31/4425

(52) U.S. Cl. CPC G01N 33/564; A61K 31/4425; G01N 2333/75; G01N 2800/28; G01N 2800/52

(57) ABSTRACT

The present invention provides for a novel method for the detection of the presence of myasthenia gravis in a subject, by means of detecting the presence of fibrinogen in serum generated from the blood of the patient. Further novel means for stabilizing the fibrinogen, for the purposes of interrogation of the sample, are provided.

### FIELD OF THE INVENTION

The present invention pertains to the field of methods of detecting and diagnosing the existence of disease in a patient.

### BACKGROUND OF THE INVENTION

All of the publications, patents and patent applications cited within this application are herein incorporated by reference in their entirety to the same extent as if the disclosure of each individual publication, patent application or patent was specifically and individually indicated to be incorporated by reference in its entirety.

Myasthenia gravis (MG) is a humoral autoimmune disorder in which autoantibodies directed against neuromuscular junction (NMJ) proteins affect the electrical signal transmission across the NMJ, resulting in variable weakness of voluntary muscles ranging from mild ocular and/or limb muscle weakness to fulminant life threatening myasthenic crises due to weakness of swallowing and breathing muscles. As a result of the nonspecific symptoms, there still remain reported challenges concerning diagnosis in the emergency room and in elderly populations with comorbid illness.

The prevailing thought is that autoantibodies in MG are directed against the nicotinic acetylcholine receptor (AChR) at NMJ, which is observed in about 85% of patients. A smaller proportion of patients have autoantibodies against other NMJ proteins, including the muscle specific tyrosine kinase (MuSK) or low-density lipoprotein receptor-related protein 4 (LRP4). No antibodies can be detected in about 5-10% of patients with generalized MG and about 50% with ocular MG, using the current assays (seronegative MG) though such patients manifest clinical features and therapeutic responses similar to those with detectable autoantibodies. Although relatively specific for the diagnosis and subgrouping of MG, the serum level of anti-AChR, anti-MuSK or anti-LRP4 antibodies do not correlate with the disease course or treatment outcomes. Limitations of these current molecular diagnostic methods are particularly exemplified with the number of seronegative patients who test negative for the ocular form (Class I) of this disease.

The identification of robust serological biomarkers in MG has previously been attempted. In one study, the blood levels of proliferation-inducing ligand and several cytokines were found to be upregulated in the sera of MG patients as compared to controls. A second study has reported correlations between MG and the serum levels of matrix metalloproteinase, transforming growth factor alpha and receptor for advanced glycation end-products binding protein. Most recently, high κ-free light chain has been reported for seropositive and seronegative forms of MG.

Although these studies provide promising results, the limited dynamic range (less than twofold) of the proteins between controls and individuals with MG leads to significant challenges for their utility in robust diagnostic testing. Additionally, it is not clear whether the elevation of serum inflammatory proteins in MG patients is disease specific or represents a non-specific, general increase in the inflammatory mediators expected in autoimmune disease. Notably, patients with MG have increased risk of having another autoimmune disorder, with about 13-22% having a second autoimmune disorder. In line with these challenges, a recent report has identified a panel of five serum metabolites, which include three lysophospholipids, glyceric acid and 12-ketodeoxycholic acid, that are reported to differentiate between MG and another autoimmune disease Rheumatoid arthritis (RA). However, again the limited dynamic range of these metabolites, of less than twofold, impairs their utilization for diagnostic testing.

The art is in need of methods based on serum proteomic biomarkers providing universal diagnosis for classes and serotypes of MG.

### SUMMARY OF THE INVENTION

In one aspect, the present invention provides for a method of diagnosing whether a human subject has Myasthenia Gravis, comprising measuring the level of fibrinogen in a blood sample from the subject; and comparing said level to the level of a reference sample from a patient without Myasthenia Gravis, wherein an increase in the level of fibrinogen relative to said reference indicates the subject has Myasthenia Gravis.

In another aspect the present invention provides for a method for diagnosing whether a human subject has Myasthenia Gravis, comprising obtaining a blood sample from the patient; preparing a serum sample from the blood sample of the patient; detecting the presence of fibrinogen in the serum sample wherein the presence of fibrinogen in the serum indicates the subject has Myasthenia Gravis. In one embodiment, detecting the presence of fibrinogen in the serum sample is by use of an immunochromatographic assay. In a further embodiment the immunochromatographic assay is a lateral flow device. In a further embodiment the antibody used in said lateral flow device has affinity for fibrinogen. In a still further embodiment, the fibrinogen is selected from the group comprising Fibrinogen-α, Fibrinogen-β, and Fibrinogen γ. In another embodiment the immunochromatographic assay is a vertical flow device. In a further embodiment the antibody used in said vertical flow device has affinity for fibrinogen. In a still further embodiment the fibrinogen is selected from the group comprising Fibrinogen-α, Fibrinogen-β, and Fibrinogen γ.

In another aspect, the present invention provides for a method for preparing a sample from a patient for the purposes of detecting the presence of Myasthenia Gravis in said patient comprising collection of a sample of whole blood from said patient; allowing said sample to rest at room temperature for a period sufficient for coagulation of red blood cells to occur; removing of the coagulated red blood cells from the sample resulting in a liquid portion substantially free of red blood cells; and storage of the liquid portion substantially free of red blood cells at below 4° C. In one embodiment the coagulated red blood cells are removed by way of centrifugation. In a further embodiment the red blood cells are removed by way of centrifugation at 1,000-2,000×g for 10 minutes.

In another aspect, the present invention provides for a method of medical treatment for a patient suspected of having Myasthenia Gravis comprising measuring the level of fibrinogen in a blood sample from the subject; comparing said level to the level of a reference sample from a patient without Myasthenia Gravis; and administering to said patient a neuroreceptor stimulator if there is an increase in the level of fibrinogen in said blood sample. In one embodiment the neuroreceptor stimulator is an inhibitor of acetylcholinesterase activity. In a further embodiment the inhibitor of acetylcholinesterase activity is pyridostigmine.

In another aspect the present invention provides for a method for preparing a sample from a patient for the purposes of detecting the presence of Myasthenia Gravis in said patient comprising collection of a sample of whole blood from said patient; allowing said sample to rest at room temperature for a period sufficient for coagulation of red blood cells to occur; removing of the coagulated red blood cells from the sample resulting in a liquid portion substantially free of red blood cells; adding an anti-coagulant to the liquid portion substantially free of red blood cells; and storage of the liquid portion substantially free of red blood cells. In one embodiment the coagulated red blood cells are removed by way of centrifugation. In a further embodiment the red blood cells are removed by way of centrifugation at 1,000-2,000×g for 10 minutes. In a further embodiment the anti-coagulant is selected from the group comprising EDTA, citrate, oxalate and SDS. In a still further embodiment the EDTA is added to the serum sample to a final concentration of 3 mM to 50 mM. In another still further embodiment the citrate is added to the serum sample to a final concentration of 0.25% to 1% w/v. In another still further embodiment the oxalate is added to the serum sample to a final concentration of 0.5 mg/mL to 3 mg/mL. In another still further embodiment the SDS is added to the serum sample to a final concentration of 0.1% to 2% w/v.

In another aspect the present invention provides for a method of medical treatment for a patient suspected of having myasthenia gravis comprising measuring the level of fibrinogen in a blood sample from the subject; comparing said level to the level of a reference sample from a patient without Myasthenia Gravis; and administering to said patient a neuroreceptor stimulator if there is an increase in the level of fibrinogen in said blood sample. In one embodiment the neuroreceptor stimulator is an inhibitor of acetylcholinesterase activity. In a further embodiment the inhibitor of acetylcholinesterase activity is pyridostigmine.

In another aspect the present invention provides for a kit comprising a means for collecting of a sample of whole blood from a human patient; a tube capable of holding said blood while being subjected to centrifugation at 1,000-2,000×g for 10 minutes, and an anti-coagulant. In one embodiment the anti-coagulant is selected from the group comprising EDTA, citrate, oxalate and SDS.

### BRIEF DESCRIPTION OF THE FIGURES

FIG. 1 shows a three-way triplot of differential protein abundance observed between MG, control, and RA samples from humans;

FIG. 2 shows a heatmap representation of statistically significant proteins with their respective calculated ANOVA F-statistic shown (p<0.05);

FIG. 3 shows total protein loading of patient serum in a SDS-PAGE gel visualized by Coomassie staining (a) and samples analyzed by Western blotting with anti-Fibrinogen-α antibodies derived against the N-terminal 21-320 residues of Fibrinogen-α (b);

FIG. 4 shows Western blot analysis for Fibrinogen-α in plasma (a) and plasma and serum (b);

FIG. 5 shows the relative amount of total fibrinogen between MG or control samples;

FIG. 6 shows Western blot analysis for residual Fibrinogen-α in serum of MG patients or controls;

FIG. 7 shows Beeswarm plots of the EICs quantified for the three fibrinogens and albumin as control in 31 MG patients, 18 RA patients and 30 controls;

FIG. 8 shows the areas under the curve and their 95% confidence intervals in addition to the specificities (True Positive Rate) and sensitivities (True Negative Rate) for targeted proteomic analysis of Fibrinogen-α/β/γ of a patient cohort;

FIG. 9 shows the results of fibrinogen detection in samples of serum following incubation at room temperature; and

FIG. 10 shows stabilization of fibrinogen in serum samples following addition of anti-coagulants in accordance with the present invention.

### DETAILED DESCRIPTION OF THE PRESENT INVENTION

As used herein the term “serum” means that portion of whole blood that remains after the suspended materials have been removed. More particularly, serum is the fluid that remains after red blood cells, fibrinogen and fibrin have been removed from whole venous blood.

As used herein “plasma” means the fluid part of blood as distinguished from the suspended materials. Plasma differs from serum in that plasma contains fibrinogen component that is absent in serum. As used herein, the terms “plasma” and “serum” connote fluids that are substantially free of red blood cells.

As used herein, room temperature is defined to be approximately 18° C. to 25° C.

The present invention is based upon the novel and unexpected observation of a high abundance of fibrinogen in the blood of MG patients, which has not been reported or known in the art. The present invention is based, further, on the novel and unexpected observation of the high abundance of fibrinogen in serum of MG patients, wherein serum of human patients is generally known in the art to contain little or no fibrinogen. The novel and unexpected finding that the removal of the majority of fibrinogen from the blood as well as red blood cells and fibrin, by way of sera preparation techniques known in the art, resulted in remnant and detectable fibrinogen levels in the serum of those patients with MG, as opposed to those without MG, where the residual MG levels were sufficient for detection by various means in the art and thereby provide a novel diagnostic method for identifying a patient with MG.

The present invention is further based on the unexpected observation of the high level of fibrinogen in patient blood surviving the conversion of blood to serum following clotting; the fibrinogen thereafter being stabilized by the addition of anti-coagulation factors following the clotting/coagulation of blood. This provides a novel means to prepare a blood sample of a patient for interrogation for the presence of fibrinogen, the presence of, or increased presence of, correlating with the patient having MG.

As disclosed herein, the heightened presence of fibrinogen in MG patient serum was detected at least 2 hours following its being obtained from patients, when stored at 4° C.; while serum from MG patients maintained at ambient room temperature did not present the high fibrinogen levels observed otherwise. Freezing and thawing of the serum also reduced the detectable levels of fibrinogen in the serum. It is therefore contemplated, as part of the present invention, that the temperature of patient serum to be tested for the presence of high fibrinogen levels identified herein as being associated with MG in a patient, be maintained at below room or ambient temperature, in a preferred embodiment under 8-10° C., in an even more preferred embodiment at or below 4° C.

As disclosed herein, addition of anti-coagulation factors following coagulation stabilizes the fibrinogen in the serum sample, allowing the serum sample to be stored for prolonged periods at temperatures above 4° C.; or subjected to one or more freeze-thaw cycles. The present invention contemplates the use of anti-coagulation compounds as generally known in the art, and in particular Ethylenediaminetetraacetic acid (EDTA), citrate, and oxalate; added to the serum sample following coagulation; and more particularly at final concentrations of 3-50 mM (EDTA), 0.25%-1.0% w/v (citrate) and 0.5 mg/mL-3 mg/ml (oxalate) in the serum, post-coagulation, are contemplated as sufficient to stabilize fibrinogen in the serum for subsequent interrogation. The present invention contemplates use of detergents as anti-coagulants, such as sodium dodecyl sulphate (SDS); wherein the addition of the detergent as an anti-coagulant is compatible with subsequent means used for detection of fibrinogen. More particularly final concentrations of 0.1%-2% w/v of SDS in the serum, post-coagulation, are contemplated as sufficient to stabilize fibrinogen in the serum for subsequent interrogation.

The present invention contemplates the use of an immunochromatographic assay, as more generally known in the art as a lateral flow test; for the detection of the presence of high amounts of fibrinogen in the serum of a patient, the presence of high fibrinogen in the serum, as demonstrated in said lateral flow test, correlating with the patient suffering from MG. In a preferred embodiment, blood sample is removed from a human patient, the blood sample is processed by means known in the art to provide a sample of human serum, and said serum is then interrogated for the presence of fibrinogen by means known in the art, and the presence of high fibrinogen correlating to the presence of MG in the patient.

The processing of human blood to provide for serum, representing the process of removal of red blood cells, fibrinogen and fibrin; while sufficient to remove red blood cells, is insufficient in MG patients to remove enough fibrinogen to provide for undetectable levels of same when using standard techniques available known to detect fibrinogen. The present invention contemplates use of a variety of quantitative and qualitative techniques for measuring the presence or amount of fibrinogen in serum including western blot, immunochromatographic assay, assays which identify the activity of fibrinogen (for example by measuring the increase of a reaction product or decrease of a reactant within the test sample), immunoassays which detect the presence of an antigenic portion of fibrinogen.

The novel and unexpected observation of high fibrinogen in MG patient serum, as further described herein, leads directly to the ability to use such elevated levels of fibrinogen, quantitatively identified in some MG patients as high as an order of magnitude greater than normal controls; as a diagnostic tool. This allows for improved capability to assess the presence of the MG disease state in a patient; and the same is contemplated as part of the present invention. It is contemplated that the invention, as described herein, provides for such diagnostic tools, along with providing additional means diagnosing the presence of MG in a patient, lower cost and less burdensome testing for MG in a patient. It is further contemplated that the present invention can provide for improved methods for detecting the otherwise undiagnosed presence of MG in a patient, and for detecting a predisposition to the development of MG in a patient.

### EXAMPLE 1: COLLECTION AND PROCESSING OF HUMAN SERUM SAMPLES

Sera samples were obtained from Canadian BioSample Repository (CBSR), collected and stored in a de-identified manner. Approval to use the sera, to investigate potential biomarker(s), was obtained from the Research Ethics Board, University of Alberta.

For analysis, three groups of samples were identified: Myasthenia gravis (MG), Rheumatoid Arthritis (RA) as a reference disease and normal controls. A total of 79 samples were recruited for the study: 31 MG, 18 RA and 30 controls. The demographics of the individuals are summarized in Table 1. MG and RA serostatus was confirmed with antibody testing for either anti-AChR/anti-MusSK (MG) or RA. RA patients were diagnosed in accordance with the American Rheumatology Association 1987 criteria (Arnett F C, et al. Arthritis Rheum. 1988;31(3):315-24). To exclude the confounds of race, only Caucasian patients were included. There were no smokers and no statistically significant differences between all groups from time of last meal or BMI. Further, patients had no history of any other autoimmune disease or thymoma. Finally, due to the nature of recruitment, patients were not required to fast. Clinical patients and healthy controls were enrolled in a prospective observational trial to obtain serum. MG and HC were collected within the same clinic. RA samples were collected in multiple clinics.

TABLE 1
Cohort Demographics.

| Myasthenia Gravis | n = 31 |
|---|---|
| Average Age (years) | 59.0 ± 23.9(range 19-93) |
| Gender (M/F, %) | 51.6%/48.4% |
| Average On-set Age | 54.7(range 15-86) |
| Early/Late On-set (%) | 35.5%/64.5% |
| Disease Severity (MGFA) | |
| Class I | 25.8% |
| Class II | 32.3% |
| Class III | 41.9% |
| Control | n = 30 |
| Average Age (years) | 48.2 ± 17.1(range 21-86) |
| Gender (M/F, %) | 40/60 |
| Rheumatoid Arthritis | n = 18 |
| Average Age (years) | 65.1 ± 11.7(range 36-81) |
| Gender (M/F, %) | 33.3/66.7 |

For collection, blood samples were drawn from the antecubital vein using a 21 G needle and vacutainer red top no additive tubes (Becton Dickenson). Demographic information and clinical profiles of the three groups of individuals from which sera were collected for fibrinogen protein analysis. As per the Myasthenia Gravis Foundation of America classification, Class I represents purely ocular from of MG, Class II represents mild generalized MG and Class III describes moderately severe generalized MG.

SDS-PAGE loading buffers contained 100 mM of β-mercaptoethanol and 4% SDS. 5 μl of samples were resolved by 10% SDS-PAGE and either visualized by Coomassie R250 staining or transferred to a nitrocellulose membrane for Western blot analysis as previously described. The membranes were then immunoblotted with either a mouse anti-FGA antibody (A-6) (SC-166968; Santa Cruz Biotechnology) derived against residues 750-850, or a rabbit anti-FGA antibody (ab92572, Abcam) derived against residues 21-320. Secondary blotting was achieved with either goat anti-Mouse or donkey anti-Rabbit IRDye 680RD labeled antibodies (LI-COR).

Coomassie stained gels and Western blots were visualized with a LiCOR Odyssey Fc system. Serum fibrinogen was quantified using an enzyme-linked immunosorbent assay (ELISA) was performed with a kit (ab208036, Abcam) following the manufacturer's recommended procedure.

18 randomly selected samples, 6 from each group, were used for the initial shotgun proteomic analysis with sample resolved by 10% SDS-PAGE and visualized by Coomassie staining. Each lane was excised into 26 bands, then each band was subjected to in-gel tryptic digestion as previously known in the art (Khan S R, et al. Chem Biol Interact. 2015;239:129-38). The resulting digested peptides were vacuum dried and re-suspended in solvent A (5% Acetonitrile, ACN, in 0.2% formic acid) for LC-MS/MS analysis. LC-MS/MS analysis was performed on an Thermo Scientific EASY-nL 1000 system inline Q-Exactive Hybrid Quadrupole-Orbitrap Mass Spectrometer using identical parameters as described by Priagasam et al. (Piragasam R S, et al. Biochem Cell Biol. 2020;98(1):61-9.) with the alteration of running a 75 min gradient.

Raw data files of MS/MS spectra for each sample were combined as searched with Proteome Discoverer 1.4.1.14 (Thermo Fisher Scientific, US) against the non-redundant and reviewed proteome of Homo sapiens retrieved from UniProt. The search parameters used were as previously described (Kramer D A, Proteomics. 2017;17(12)).

Extracted ion chromatograms (EICs) were used to quantify the protein abundance in individual samples, for which the values were subjected to normalization by the proportion to total ion current (TIC) as previously described (Kramer D A, Proteomics. 2017;17(12)). One-way ANOVA was employed for comparative analysis of the three sample groups, using a statistical cut-off of a p<0.05.

Serum proteins were resolved by 8% SDS-PAGE then prepared for LC-MS/MS as described above. The resulting fractions from each sample were then pooled prior to LC-MS/MS analysis. Multiple peptides per protein were included for method building and final quantification using the Skyline software package were implemented as described by Priagasam et al. (Piragasam RS, et al. Biochem Cell Biol. 2020;98(1): 61-9.).

### EXAMPLE 2: PROTEOME PROFILING OF HUMAN SERUM SAMPLES

To identify novel proteins in an unbiased manner that may be specifically associated with MG, a pilot study using a gel-based shotgun proteomics method was applied to the analysis of unadulterated serum samples collected from six healthy controls, six MG patients and six RA patients. A total of 502 proteins were identified with the criteria of two or more quantifiable peptides per protein to limit the false discovery rate (FDR) of protein identifications. Within the MG group 361 proteins were identified, while for the control and RA groups, 327 and 304 were identified respectively. Of these proteins, 212 were common to all groups.

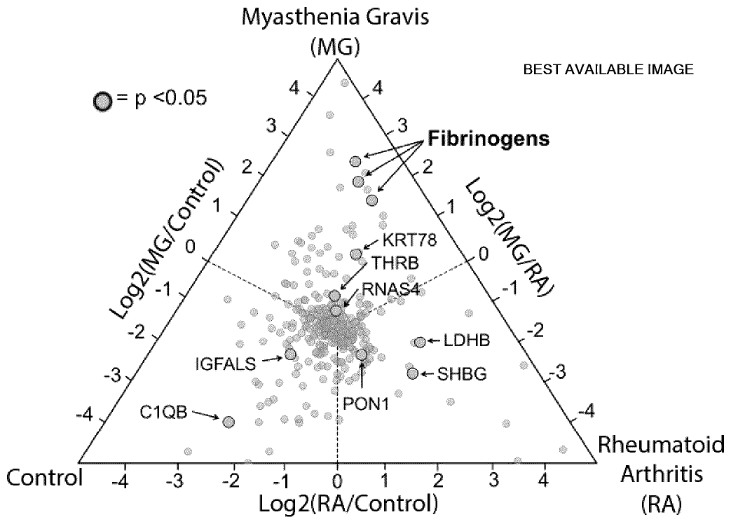

The ratios of the average relative extracted ion intensity for each protein compared among all three test groups are presented in FIG. 1 which shows the clustering of the data points in the center of the graph revealing that the majority of average ion intensities for each protein is similar between all three groups, but that some proteins are higher in some of the groups with the data being closer to the triangle apex from the respective group. The log₂ ratio of the normalized protein abundance was plotted to visualize differential amounts between the three groups, with proteins observed in higher abundance in one group over others observed closer to the respective vertices. As some average ion intensities may be biased as a result of highly variable measurements from individuals in a group, either from technical variability in quantification or true variability of a protein in an individual, all the data was analyzed using a one-way ANOVA to identify proteins that exhibit statistically significant differences between the MG, RA and control groups. The proteins that met the criteria of a P<0.05 are identified by arrows and names FIG. 1. While this cut-off may lead to false-positive identifications as a result of the multiple testing problem it does warrant further investigation of these identified proteins.

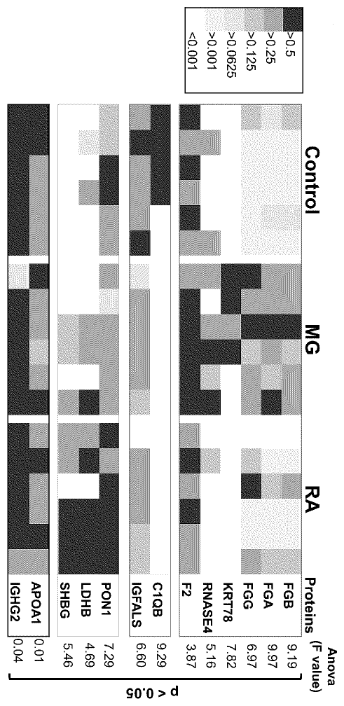

The individual measurements of the 11 proteins that exhibited statistically significant differences between the MG, RA and control groups are summarized in the heat map shown in FIG. 2 along with Apolipoprotein A1 (APOA1) and the constant region of immunoglobulin heavy chain (IGHG2) as controls. In addition, the corresponding ANOVA F-statistic and P-values are also listed for each protein. The data reveals that fibrinogen-α (FGA), fibrinogen-β (FGB), fibrinogen-γ (FGG), keratin, type II cytoskeletal 78 (KRT78), ribonuclease 4 (RNASE4) and prothrombin are consistently in higher abundance in MG serum. Serum paraoxonase/arylesterase 1 (PON1), L-lactate dehydrogenase B (LDHB), and sex hormone-binding globulin (SHBG) are consistently high in RA sera, while complement C1q subcomponent subunit B (C1QB) and insulin-like growth factor-binding protein complex acid-labile subunit (IGFALS) are low in both MG and RA sera.

To further narrow the focus of the investigation, proteins consistently exhibiting the largest observed differences were selected for further investigation. As a control for the quantification by extracted ion intensities. The quantified data for the control proteins, including several immunoglobulins, apolipoproteins, albumin and C4b-binding protein alpha chain, revealed the spread in the quantified data for all three groups (MG, RA, and controls). For the proteins observed in differential abundance, all three fibrinogen subunits (FGA, FGB and FGG) exhibited the largest differential abundance in MG patient sera. The amounts of these fibrinogens were consistently higher in abundance by over an order of magnitude compared to controls (p<0.01) and were thus selected for further biochemical validation.

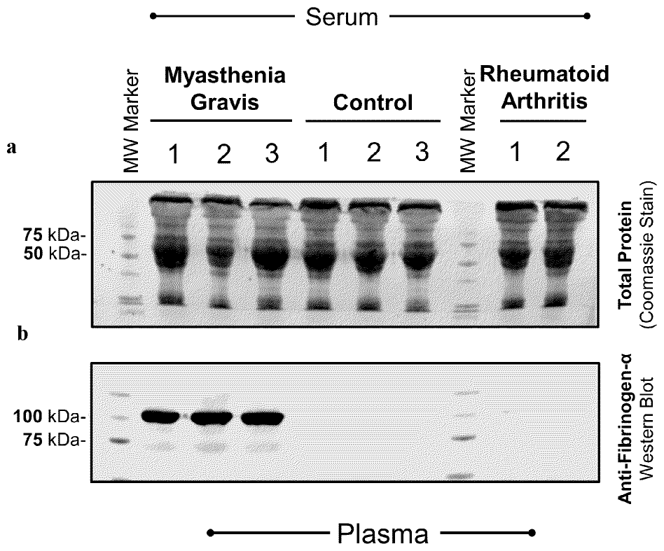

To validate the observations of high serum fibrinogen levels in MG patients, serum samples from MG, RA and control individuals, were analyzed for Fibrinogen-α by immunoblotting with samples normalized to total protein loading as visualized by Coomassie Blue staining, to ensure equal protein loading for analysis. Immunoblotting for Fibrinogen-α, as presented in FIG. 3a, revealed a very high protein amounts in MG sera, which was essentially undetectable in either the RA or control samples. The epitope for this anti-Fibrinogen-α antibody is mapped to the amino acid residues 750-850, so this large differential detection of Fibrinogen-α was further validated by using an anti-Fibrinogen-α antibody derived against the residues 21-320 (FIG. 3b), that resulted in essentially the same results in high levels only being observed in sera from MG patients. This second antibody for the N-terminal region of fibrinogen revealed altered apparent gel mobilities for Fibrinogen-α, suggesting that proteolytic processing is occurring that was not apparent by the analysis with the C-terminal specific antibody. Of note, the C-terminal specific antibody was used in all additional immunoblotting experiments.

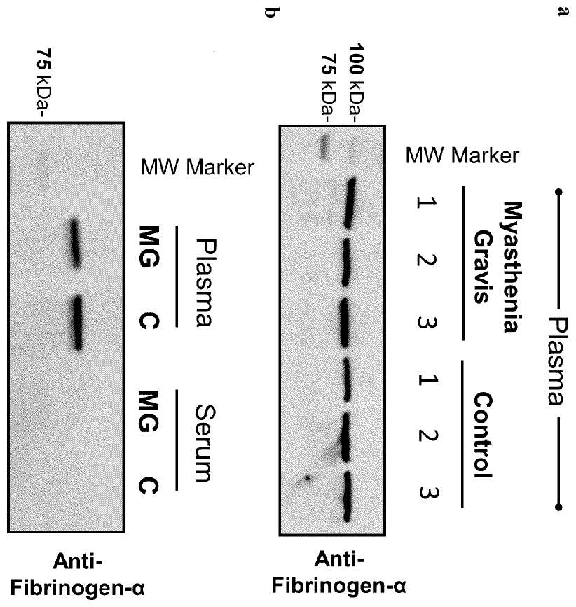

Fibrinogen-α amounts in the plasma of MG and controls were investigated by immunoblotting (FIG. 4a). The analysis revealed no significant difference in Fibrinogen-α in MG plasma when compared to a control sample. To investigate whether Fibrinogen-α was being removed during the collection and preparation of patient serum, both the plasma and serum of an MG patient and a control sample were analyzed simultaneously (FIG. 4b). What is observed is that when analyzing plasma, the Fibrinogen-α in serum is undetectable as a result of the limited dynamic range of Western blotting. Only when analyzing serum alone, is the high level of residual Fibrinogen-α in the serum of MG patients observable (FIG. 3b).

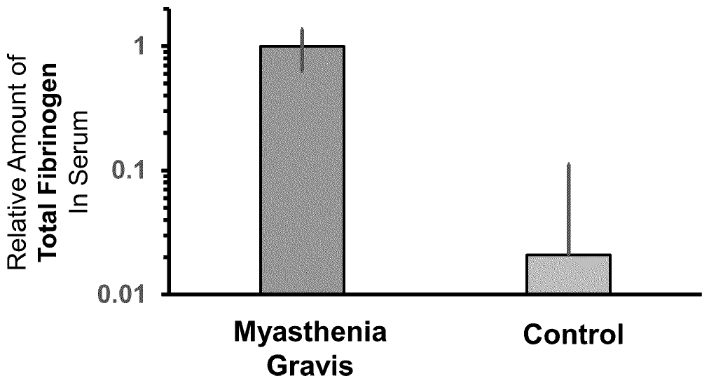

An ELISA assay for total fibrinogen was performed, in part, due to the limited dynamic range of detection by way of immunoblotting. Due to the unexpectedly large differences in fibrinogen detected, the MG serum samples required dilution in order for them to be on the same linear range of detection for the assay when comparing to control samples. The resulting data from the ELISA is shown in FIG. 5, and consistent with proteomic analysis, revealed over an order of magnitude difference when comparing total fibrinogens in MG and control sera.

To investigate the stability of the observed residual fibrinogen in the MG patient sera, the presence of fibrinogen was investigated upon prolonged incubations. Freshly thawed MG and control serum samples (T=0) were further incubated for 2 hours at either 4° C. or at ambient room temperature (˜20° C.). Fibrinogen in the samples was then detected by immunoblotting with an anti-Fibrinogen-a antibody; and while the residual fibrinogen persisted at low temperature, it was no longer detectable upon incubating at room temperature, indicating the transient nature of the residual serum fibrinogen.

### EXAMPLE 3: BLINDED ANALYSIS OF RESIDUAL SERUM FIBRINOGEN IN MG PATIENTS

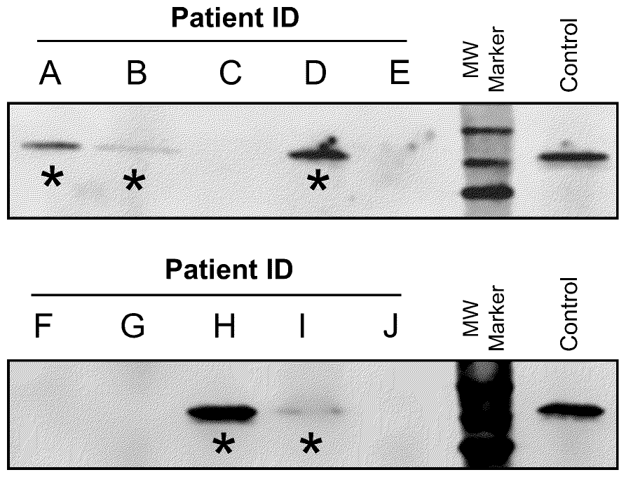

A blinded study was initiated to verify whether the correlation of serum fibrinogen and MG held true with a new set of blinded samples. A total of 10 blinded sera samples (A-J) were obtained from the MG clinic and analyzed for Fibrinogen-a by Western blotting. The resulting analysis revealed protein bands corresponding to Fibrinogen-α for samples A, B, D, H and I (FIG. 6). Unblinding of the clinical data revealed a 100% identification rate for MG patients from normal controls (patients C, E, F, G & J).

### EXAMPLE 4: COHORT ANALYSIS BY TARGETED MASS SPECTROMETRY FOR RESIDUAL SERUM FIBRINOGEN

With the promising findings of the initial proteomic study with regards to the correlation of fibrinogens and MG diagnosis along with the result of the blinded study with fibrinogen-α (FIG. 6) a validation study was conducted using a larger patient cohort consisting of sera from 31 MG patients (27 anti-AChR antibody positive, one anti-MuSK antibody positive, three seronegative), 18 from RA patients and 30 controls. The demographic information and clinical profiles for this cohort are as outlined in Table 1.

To circumvent the challenges of the limited dynamic range of detection observed for Western blot and ELISA assays, the serum samples were digested with trypsin and simultaneously analyzed for the tryptic peptides derived from fibrinogen-α, fibrinogen-β, fibrinogen-γ and serum albumin (as a control) by parallel reaction monitoring (PRM). PRM is a targeted mass spectrometry technique that results in reduced analytics variability compared to the label-free shotgun proteomic analysis used in the initial identification of the fibrinogens as possible targets for identifying the disease state. For PRM analysis of 3 peptides for fibrinogen-α, 4 peptides fibrinogen-β, and 5 fibrinogen-γ were targeted for quantification; along with four peptides for the serum albumin control. Extracted ion chromatograms (EICs) of minimum five co-eluting fragment ions were measured for the quantification for each peptide, while being verified by aligning the retention time with the precursor ion for the given peptide to eliminate any possible mass spectrometry artifacts and quality assurance of the identified fragment ions. By definition, the quantified abundance of each protein was the sum of EICs of corresponding peptide fragment ions normalized to Serum Albumin.

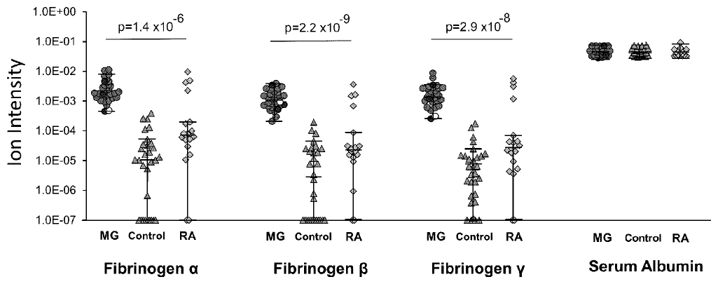

The quantification of fibrinogen-α, -β and-γ in the 79 serum samples by PRM analysis, shown in the Beeswarm plots in FIG. 7, clearly reveals substantially equally as high abundance of all three fibrinogens in MG patient sera when contrasted to either control or RA serum samples. Of note, the sera from three seronegative and one anti-MuSK MG patients were indistinguishable from the other MG patients with regards to all three fibrinogens. While the majority of control and RA samples exhibited a significantly lower abundance of the fibrinogens, few individuals did reveal high fibrinogen levels. Within the dynamic range of detection of about five orders of magnitude with the PRM analysis, the median values for the three fibrinogens in MG samples were 28 to 54-fold higher than the RA samples and 250 to 4000-fold higher than the control samples. The MG sera revealed about a 10-fold variation among individuals for the three fibrinogens, while the controls and RA sera exhibited about a 1000-fold variation. Nonetheless, one-way ANOVA analysis revealed high degrees of statistical confidence for fibrinogen-α, fibrinogen-β and fibrinogen-γ with P values of 1.4×10⁻⁶, 2.2×10⁻⁹ and 2.9×10⁻⁸, respectively. With the similar patterns of outliers for all the fibrinogens observed (FIG. 7), the correlations of fibrinogen-α, fibrinogen-β and fibrinogen-γ abundance in all patients were compared, relating fibrinogen-α abundance in an individual against either fibrinogen-β or fibrinogen-γ, which showed significant multicollinearity among the proteins. The observation of a lack of fibrinogens acting as independent variables is consistent with each of fibrinogen fibrinogen-α, fibrinogen-β and fibrinogen-γ being subunits of the larger fibrinogen complex.

To exclude the observed fibrinogen levels as arising from treatment regimens of the patients, rather than the MG disease state; clinical records of the MG cohort were investigated with respect to the current and past drugs used by individual patients.

The diversity in the drug regiments of these individuals and the occurrence of high residual fibrinogen in some controls and individuals with RA led us to conclude that these treatments are not the source of high residual fibrinogen.

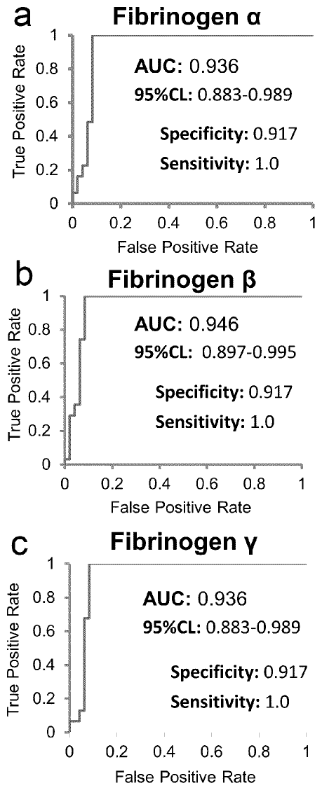

With the observed high statistically confident association between MG and residual serum fibrinogen, the data from the PRM analysis was applied to the receiver operator curve (ROC) analysis. For the ROC analysis, the data from the MG patients were compared to the combined pool of both RA and controls. The results of the targeted proteomic analysis of Fibrinogen-α/β/γ of the patient cohort were analyzed for each Fibrinogen form indicated using ROCs comparing MG patients (n=31) and the combined population (n=48) of controls and RA patients. The resulting ROC analysis for fibrinogen-α, fibrinogen-β and fibrinogen-γ are individually shown in FIG. 8. ROC analysis was not performed combinatorially with the three fibrinogens as a result of the multicollinearity of these proteins. The areas under the curve and their 95% confidence intervals in addition to the specificities (True Positive Rate) and sensitivities (True Negative Rate) for each protein are provided in their respective inserts.

To highlight the utility of the novel observation of the large magnitude of the differences in serum fibrinogens for diagnosis of MG, arbitrarily reducing the cut-off for assignment for high fibrinogens to ten-fold below that for the lowest observed case for a MG sample for ROC analysis; reduced the accuracy of predicting MG to only 75.9%, while the sensitivity remains at 100%. In contrast, the quantification of inflammatory proteins and metabolites, as known in the art, only identified a 2-fold difference between MG and the control groups.

### EXAMPLE 5: STABILIZATION OF FIGRINOGEN FOLLOWING COAGULATION OF BLOOD SAMPLE

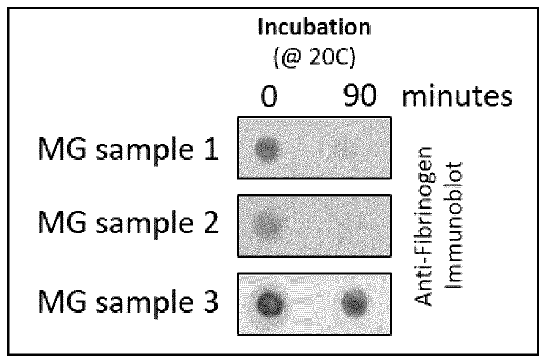

As shown in FIG. 9, incubation of serum samples at 20° C. following coagulation can lead to fibrinogen degradation, destruction or conversion to a biomolecule not detectable as fibrinogen using the methods disclosed herein; all of which renders the fibrinogen undetectable to interrogation using anti-fibrinogen antibodies. Further, this inability to detect fibrinogen on interrogation of the sample is not consistent between patients. Freezing and thawing of serum can also inhibit detection of fibrinogen remaining in the samples following conversion of the blood to serum; similar to incubation at 20° C.

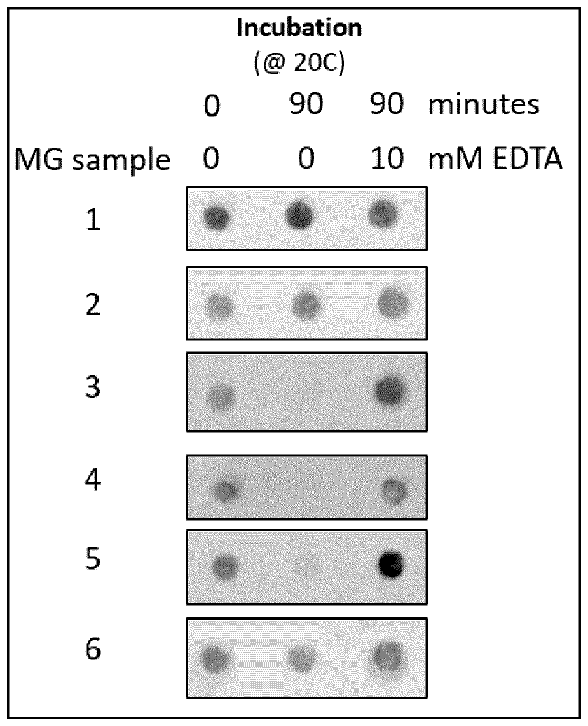

FIG. 10 shows the results of anti-fibrinogen antibody immunoblots following incubation at incubation at 20° C., with or without addition of anti-coagulants, specifically EDTA. As shown in FIG. 10, addition of anti-coagulation factors following coagulation of the blood sample stabilizes the fibrinogen for detection, allowing it to be interrogated even after at 20° C., or subject to freeze-thaw cycles (not shown).

While particular embodiments of the present invention have been described in the foregoing, it is to be understood that other embodiments are possible within the scope of the invention and are intended to be included herein. It will be clear to any person skilled in the art that modifications of and adjustments to this invention, not shown, are possible without departing from the spirit of the invention as demonstrated through the exemplary embodiments. The invention is therefore to be considered limited solely by the scope of the appended claims.

What is claimed is:

1. A method of diagnosing whether a human subject has Myasthenia Gravis, comprising:
measuring the level of fibrinogen in a blood sample from the subject; and
comparing said level to the level of a reference sample from a patient without Myasthenia Gravis, wherein an increase in the level of fibrinogen relative to said reference indicates the subject has Myasthenia Gravis.

2. A method for diagnosing whether a human subject has Myasthenia Gravis, comprising:
Obtaining a blood sample from the patient;
Preparing a serum sample from the blood sample of the patient;
Detecting the presence of fibrinogen in the serum sample wherein the presence of fibrinogen in the serum indicates the subject has Myasthenia Gravis.

3. The method of claim 2 wherein detecting the presence of fibrinogen in the serum sample is by use of an immunochromatographic assay.

4. The method of claim 3 wherein the immunochromatographic assay is a lateral flow device.

5. The method of claim 4 wherein the antibody used in said lateral flow device has affinity for fibrinogen.

6. The method of claim 5 wherein the fibrinogen is selected from the group comprising Fibrinogen-α, Fibrinogen-β, and Fibrinogen γ.

7. The method of claim 3 wherein the immunochromatographic assay is a vertical flow device.

8. The method of claim 7 wherein the antibody used in said vertical flow device has affinity for fibrinogen.

9. The method of claim 8 wherein the fibrinogen is selected from the group comprising Fibrinogen-α, Fibrinogen-β, and Fibrinogen γ.

10. A method for preparing a sample from a patient for the purposes of detecting the presence of Myasthenia Gravis in said patient comprising:
Collection of a sample of whole blood from said patient;
Allowing said sample to rest at room temperature for a period sufficient for coagulation of red blood cells to occur;
Removing of the coagulated red blood cells from the sample resulting in a liquid portion substantially free of red blood cells; and
Storage of the liquid portion substantially free of red blood cells at below 4° C.

11. The method of claim 10 wherein the coagulated red blood cells are removed by way of centrifugation.

12. The method of claim 11 wherein the red blood cells are removed by way of centrifugation at 1,000-2,000×g for 10 minutes.

13. A method of medical treatment for a patient suspected of having myasthenia gravis comprising:
measuring the level of fibrinogen in a blood sample from the subject;
comparing said level to the level of a reference sample from a patient without Myasthenia Gravis; and
administering to said patient a neuroreceptor stimulator if there is an increase in the level of fibrinogen in said blood sample.

14. The method of claim 13 wherein the neuroreceptor stimulator is an inhibitor of acetylcholinesterase activity.

15. The method of claim 14 wherein the inhibitor of acetylcholinesterase activity is pyridostigmine.

16. A method for preparing a sample from a patient for the purposes of detecting the presence of Myasthenia Gravis in said patient comprising:
Collection of a sample of whole blood from said patient;
Allowing said sample to rest at room temperature for a period sufficient for coagulation of red blood cells to occur;
Removing of the coagulated red blood cells from the sample resulting in a liquid portion substantially free of red blood cells;
Adding an anti-coagulant to the liquid portion substantially free of red blood cells; and
Storage of the liquid portion substantially free of red blood cells.

17. The method of claim 16 wherein the coagulated red blood cells are removed by way of centrifugation.

18. The method of claim 17 wherein the red blood cells are removed by way of centrifugation at 1,000-2,000×g for 10 minutes.

19. The method of claim 18 wherein the anti-coagulant is selected from the group comprising EDTA, citrate, oxalate and SDS.

20. The method of claim 19 wherein the EDTA is added to the serum sample to a final concentration of 3 mM to 50 mM.

21. The method of claim 19 wherein the citrate is added to the serum sample to a final concentration of 0.25% to 1% w/V.

22. The method of claim 19 wherein the oxalate is added to the serum sample to a final concentration of 0.5 mg/mL to 3 mg/mL.

23. The method of claim 19 wherein the SDS is added to the serum sample to a final concentration of 0.1% to 2% w/v.

24. A method of medical treatment for a patient suspected of having myasthenia gravis comprising:
measuring the level of fibrinogen in a blood sample from the subject;
comparing said level to the level of a reference sample from a patient without Myasthenia Gravis; and
administering to said patient a neuroreceptor stimulator if there is an increase in the level of fibrinogen in said blood sample.

25. The method of claim 24 wherein the neuroreceptor stimulator is an inhibitor of acetylcholinesterase activity.

26. The method of claim 25 wherein the inhibitor of acetylcholinesterase activity is pyridostigmine.

27. A kit comprising a means for collecting of a sample of whole blood from a human patient; a tube capable of holding said blood while being subjected to centrifugation at 1,000-2,000×g for 10 minutes, and an anti-coagulant.

28. The kit of claim 27 wherein the anti-coagulant is selected from the group comprising EDTA, citrate, oxalate and SDS.
